(19) United States
(12) Patent Application Publication
(10) Pub. No.: US 2004/0162548 A1
(43) Pub. Date: Aug. 19, 2004

(54) Method and apparatus for excimer laser ablation of obstructions

(76) Inventor: Christopher Reiser, Colorado Springs, CO (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 10/368,893

(22) Filed: Feb. 18, 2003

(51) Int. Cl. A61B 18/20

(52) U.S. Cl. 606/7; 606/15

(57) ABSTRACT

A device and method for excimer laser ablation is provided wherein fibers in a catheter tip are arranged in an eccentric bundle. The optical fibers are cut and polished at an angle with respect to the end face of the catheter. The guidewire lumen lumen is preferably offset from the center of the tip.

### FIELD OF THE INVENTION

The present invention relates to a method and apparatus that employs a light source, optical fibers and catheters for use in laser angioplasty and other medical procedures.

### DISCUSSION

Excimer laser angioplasty has been studied in humans, for the purpose of opening obstructed arteries, for at least ten years. During this time, as many as five generations of laser catheters have been designed, approved by regulatory bodies, and used successfully in patients. The purpose of these catheters is to ablate or vaporize the artherosclerotic plaque and thrombus that is blocking blood flow inside the artery. Restoration of brisk blood flow is the point of these interventions as more blood flow is better. It follows that a catheter that creates a larger hole (“lumen”) in the artery has an advantage, because it lets more blood flow through that artery after treatment. This “bigger is better” philosophy has been one of the guiding principles of excimer laser coronary angioplasty (ELCA) catheter designs over the past ten years.

In a standard ELCA procedure, the ELCA catheter is threaded over a guidewire, up to the lesion, so that the fibers at the distal end of the catheter contact the lesion tissue. As the laser creates pulses of light that emerge from the fibers, the tissue is slowly vaporized in front of the catheter tip. As the catheter advances through the lesion, a hole is ablated through the lesion that is approximately the same size as the ELCA catheter. When the catheter is removed, blood can flow through the lumen created by the laser ablation path of the catheter.

The overall size of ELCA catheters is constrained by the inner diameter of the guide catheter through which the ELCA catheter must slide en route to the artery blockage. In most cases, this limits the diameter of the ELCA catheter to about 0.096 inches, or just over 2 mm. Many coronary arteries are larger than this, ranging up to 4.5 mm in diameter. In such arteries, physicians desire to create lumens larger than the physical size of the ELCA catheter. This poses a set of challenges for the ELCA catheter designer.

Solutions to the problem of making a lumen larger than the size of the catheter have been patented by several inventors. Some of these solutions include use of an expandable tip and use of an integral balloon that spreads the fibers outward. After a first pass through the lesion, the catheter is pulled back, the balloon is expanded, and the catheter is passed through the lesion a second time. The final lumen diameter is presumably determined by how large the balloon can make the catheter tip appear.

Another approach uses an eccentric catheter, in which the fibers are arranged in a bundle on one side of the tip and a guidewire lumen is arranged on the other side of the tip. After a first pass through the lesion, the catheter is pulled back and rotated approximately 60-90 degrees. Then, a second pass is made. By repeating this pull-back-rotation-repeat-ablation process, lumens up to 3 mm in diameter have been reported while using a 2 mm eccentric ELCA catheter.

Recently a German physician, Dr. Johannes Dahm, describes a technique for using eccentric ELCA catheters that differs from the standard method. Dr. Dahm rotates the catheter through a 360 degree turn while advancing the catheter forward no more than a millimeter. This ablates as much of the proximal stump of the lesion as possible, without passing the catheter through the lesion initially. Very delicate pressure must be delivered to the catheter to prevent rapid forward motion of the catheter tip, while the tip is swept back and forth through the 360 degree rotations. Delivering this delicate pressure, under fluoroscopic guidance, takes remarkable skill.

Detailed examination of the eccentric catheter tip during an ablation-during-rotation procedure reveals a few deficiencies. First, since the guidewire lumen is symmetrically located on the distal face, the surface of the catheter meeting new tissue, as the catheter advances into the lesion mass while rotating, is actually the outer surface of the catheter body. There are no fibers in the outer surface, and so this surface does not contribute to the ablation process. Reducing the amount of the outer surface contacting lesion tissue can be accomplished by moving the guidewire lumen off-center in the crescent-shaped surface at the catheter tip. However, this does not totally eliminate contact between non-ablating surfaces of the catheter tip and the lesion.

The present invention is directed to overcoming, or at least reducing, the effects of one or more of the problems discussed above.

### SUMMARY OF THE INVENTION

To address these and other drawbacks, a device and method for excimer laser ablation is provided wherein fibers in a ablation tip according to the invention are arranged in a bundle at the tip of the catheter. The optical fibers are cut and polished at an angle with respect to the end face of the catheter. The guidewire lumen is preferably offset from the center of this tip.

In another embodiment, the tip is crescent shaped. The shape of the angle that the crescent-shaped surface makes with respect to the longitudinal axis of the catheter is 90 degrees. In another aspect, this angle is moved to about 70 degrees to extend one edge of the crescent ahead of the rest of the catheter. The guidewire lumen is disposed at the other edge of the crescent. This skews the bundle, so that the fibers no longer point along the axis. That is, the normal to the surface does not intersect the longitudinal axis of the catheter. As the catheter is rotated around the guidewire lumen, the thrust-forward edge contacts the lesion tissue first, exposing it to the ablating action in the D-shaped fiber bundle. In profile, this catheter tip looks unsymmetrical, and bears some similarity to machine tool bits used with a lathe.

In another aspect, a method of using the device for laser ablation is provided that includes rotating the catheter tip continuously while the laser is operating. This can be accomplished by rotating the entire catheter body after grasping the catheter around its shaft proximal to its emergence from the guide catheter assembly. To prevent twisting of the tail tube (the section of the catheter between the point of grasping and the laser connector) and wrapping of the guidewire around the catheter, a laser connector is provided that spins freely after it is plugged into the laser. Alternatively, the circular motion could be periodically stopped and reversed so as to untwist the tail tubing. In yet another alternative, the entire catheter and laser assembly can be rotated as a unit, to avoid twisting the tail tube and the guidewire.

In another aspect, a method for assembling the device is disclosed that includes the steps of placing optical fibers through a tip shell, placing an epoxy or resin in the shell and around the optical fibers, and machining the ablation face to a desired configuration.

### BRIEF DESCRIPTION OF THE DRAWINGS

The various features, advantages and other uses of the present invention will become more apparent by referring to the following description and drawings in which:

FIG. 1 is a perspective view of a catheter assembly according to the present invention;

FIG. 2 is a perspective view of a catheter tip according to the present invention;

FIG. 3A is a perspective view of a catheter tip according to the present invention;

FIG. 3B is a side view of a catheter tip according to the present invention;

FIG. 4A is a perspective view of a catheter tip according to the present invention;

FIG. 4B is a side view of a catheter tip according to the present invention;

FIG. 5A is a perspective view of a catheter tip according to the present invention;

FIG. 5B is a perspective view of a catheter tip according to the present invention; and

FIG. 5C is a perspective view of a catheter tip according to the present invention.

### DETAILED DESCRIPTION

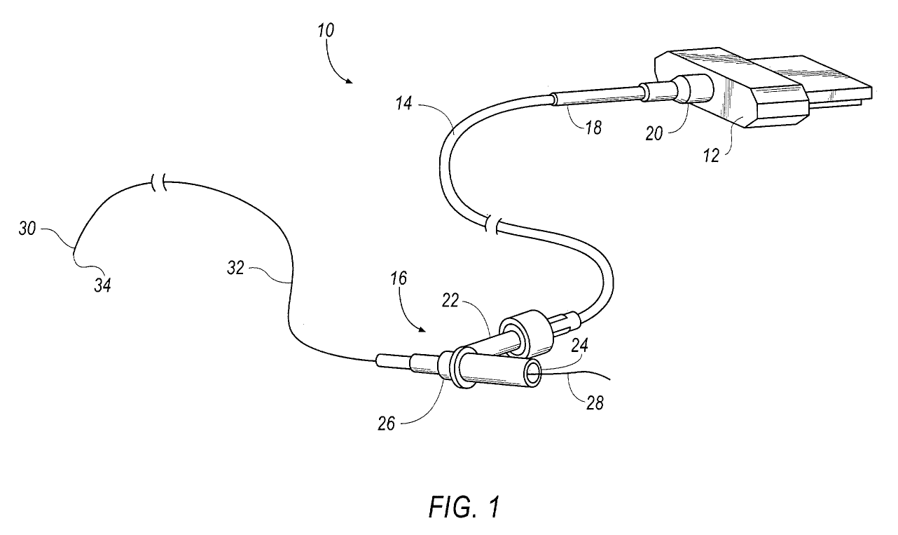

Referring now to FIG. 1, the present invention is shown and described. FIG. 1 is a perspective view of a fiber optic-catheter assembly 10 and proximal coupler 12. The proximal coupler provides an interface between the fiber optic-catheter assembly 10 and a light source (e.g., laser, not shown). The assembly 10 includes a light conveying cable 14, which contains optical fibers that direct light from the proximal coupler 12 to a bifurcating adapter 16. A proximal end 18 of the cable 14 includes a strain relief sleeve 20, which is typically made of coiled metal or an elastomer and helps reduce damage to the assembly 10 due to forces exerted on the cable 14 during handling. Note that throughout the specification the terms “proximal” and “distal” refer to the location of a component of the assembly 10 relative to the light source—a component that is nearer to the light source is “proximal,” whereas a component that is further away from the light source is “distal.”

The proximal coupler 12 may be a two-piece mount as described in U.S. patent application Ser. No. 07/899,470 to Nielson et al., incorporated herein by reference, and it may be of the linear scan type described in U.S. patent application Ser. No. 07/882,597 to Grace et al., also incorporated herein by reference.

The bifurcating adapter 16 includes a pair of branches 22, 24 that converge into a single trunk 26. One of the branches 22 receives the light conveying cable 14, while the other branch 24 receives a guide wire into a guide wire lumen 28 extending in this case from the adapter branch 24 to the distal tip 30. The guide wire is used to route the distal end 30 of the fiber optic-catheter assembly 10 from an entry point in the body to a treatment area. The trunk 26 of the bifurcating adapter 16 receives a catheter 32, which generally comprises an outer tube and an inner tube (not shown). The annular region between the outer tube and the inner tube defines an outer lumen, which contains optical fibers, and the interior of the inner tube defines an inner lumen that contains the guide wire lumen 28. The inner and outer tubes may be constructed from any of a number of suitable materials, including plasticized vinyl resins, polyethylene, polytetrafluoroethylene, synthetic and natural rubbers and polyurethane elastomers. The distal end 30 of catheter 32 terminates at a tip 34, which is adapted to deliver light to the treatment area. The catheter 32 and conveying cable 14 and attachments can be formed as any conventional or other know device, such as that disclosed in U.S. Pat. No. 5,456,6800 to Taylor et al., incorporated herein by reference.

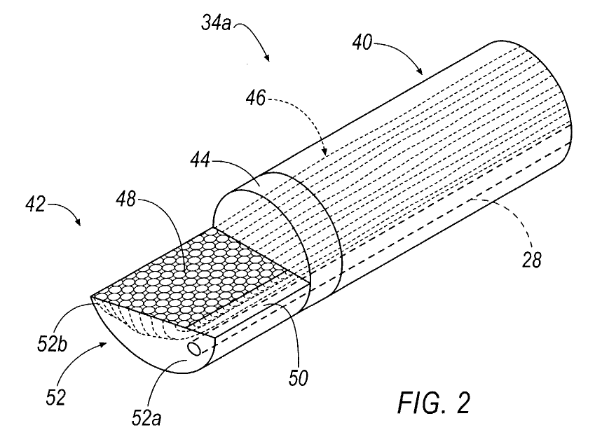

Referring now to FIG. 2, a first embodiment of tip 34a is described. Tip 34a generally includes a outer shell 40 and an ablation tip 42. Outer shell 40 is preferably cylindrical and includes marker band 44 for fluoroscopic visualization of the tip 34a. Outer shell 40 also preferably houses a plurality of optical fibers 46 from light conveying cable 14. Outer shell 40 also houses guide wire lumen 28 for guiding the tip 34a to and through a desired location.

Ablation tip 42 includes ablation face 48 and non-optical face 50. Optical fibers 46 connect to ablation face 48 such that light transmitted down optical fibers 46 exits ablation face 48. Guide wire lumen 28 connects to ablation tip 42 for guidance and positioning as will be described in greater detail. Distal face 52 is on a axially distal end of tip 34a and is generally angled from a shallow position 52a proximate guide wire lumen 28 to a deep position 52b that is distally located from guide wire lumen 28. The angle of distal face 52 can be any sufficient angle to allow increased cutting surface at a position most distal from guide wire lumen 28.

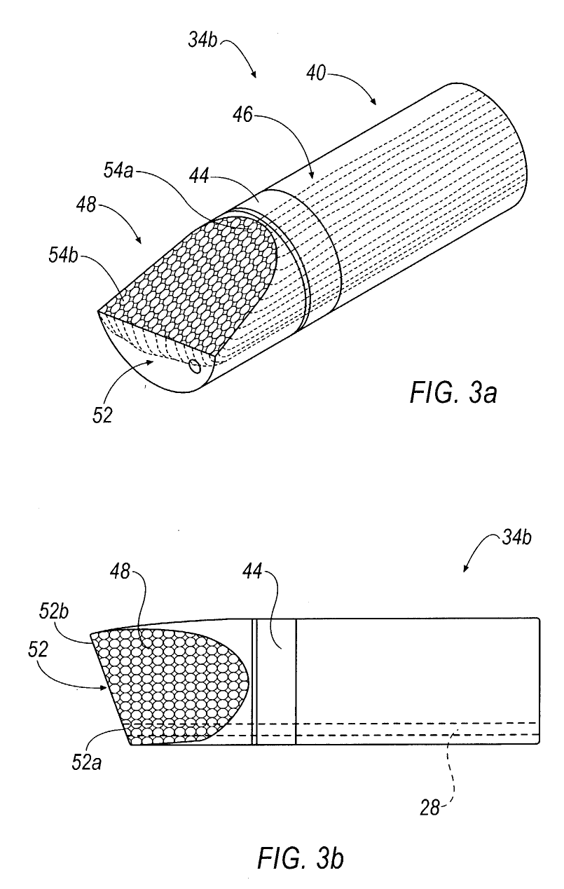

Referring now to FIGS. 3a and 3b, another embodiment of tip 34b according to the present invention is shown and described. In FIG. 3a, ablation face 48 tapers from a radial outer position 54a to a radially central position 54b. Preferably, this taper is about 15-20 degrees from the axis of the tip 34b. The angular face of ablation face 48 directs some illumination forward to ablate material slightly forward of ablation tip 42. Additionally, as in the previous embodiment, distal face 52 is on a axially distal end of tip 34a and is generally angled from a shallow position 52a proximate guide wire lumen 28 to a deep position 52b that is distally located from guide wire lumen 28. The angle of distal face 52 can be any sufficient angle to allow increased cutting surface at a position most distal from guide wire lumen 28.

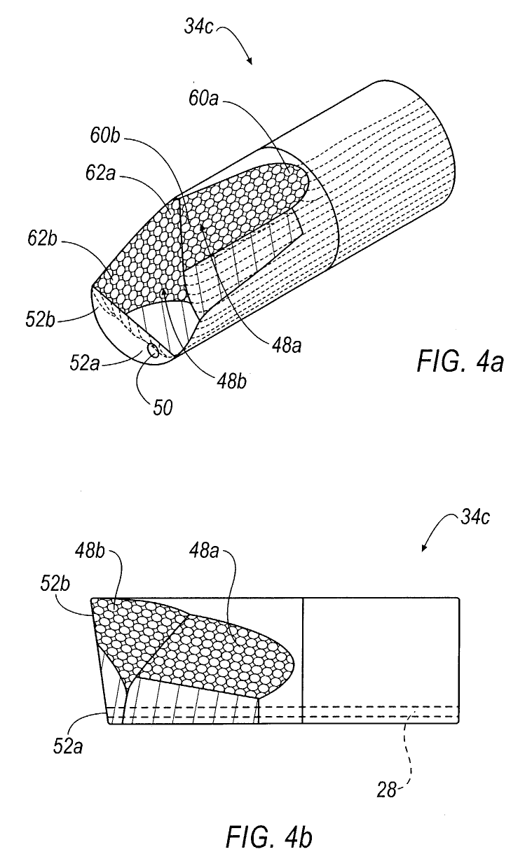

Referring now to FIGS. 4a and 4b, a third embodiment of tip 34c is shown and described. In FIG. 4a, the ablation face includes ablation surfaces 48a and 48b. Ablation surface 48a tapers from a radially outward position 60a to a more radial center position 60b. Likewise, ablation surface 48b tapers from a radially outward position 62a to a radially inward position 62b. Preferably, the taper angle of ablation surface 48a is less than ablation surface 48b. The steeper angle of ablation surface 48b allows the axially distal portion of tip 34c to project illumination forward of tip 34c as well as off to a side. As in the previous embodiments, Distal face 52b is on a axially distal end of tip 34c and is generally angled from a shallow position 52a proximate guide wire lumen 28 to a axially deep position 52b that is distally located from guide wire lumen 28. The angle of distal face 52b can be any sufficient angle to allow increased cutting surface at a position most distal from guide wire lumen 28. It should be noted that, although the embodiments show an angling of the distal face, such is not necessary for the operation of the invention, and is therefore a preferred embodiment.

Referring now to FIGS. 1-4, the operation of the present invention is described. Any of the tips 34, 34a, 34b or 34c are positioned proximate artherosclerotic plaque and thrombus or other known blockage, by means well known and understandable to one skilled in the art, such that ablation face 48 is positioned as close as possible to the blockage. Next, the catheter tip is rotated to cause rotation of the tip about the guide wire lumen 28. This causes the ablation face 48 to sweep about an axis of the guide wire and impact blockages and to vaporize those blockages with directed illumination. As the deep axial position 52b of the tip is at the axially most deep portion as well as the radially most outward position, ablation face 48 proximate to deep position 52b is first to penetrate any blockage. Alternatively, it is understood that the assembly 10 can be rotated about the axis of branch 24. This rotation causes rotation similar to that above. The rotation of either assembly 10 or guide wire lumen 28 can either be by hand, or may be mechanized by stepping motor, electric motor or other known driving means.

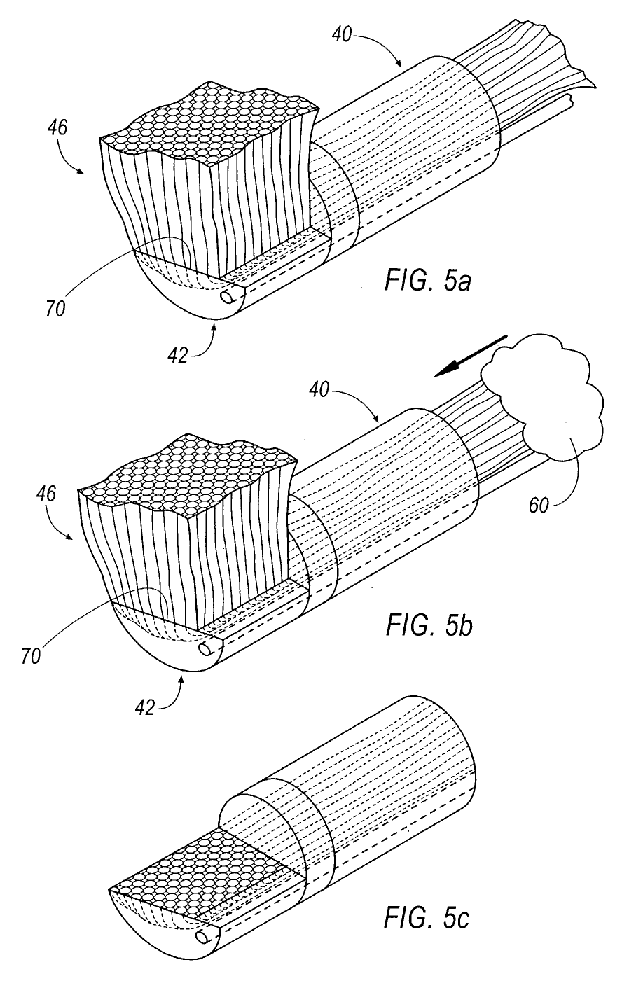

Referring now to FIG. 5, the assembly of the present invention is shown and described. In step 5a, a bundle of optical fibers 46 is positioned through outer shell 40. Next, in FIG. 5b, epoxy or other similar substance is injected into the shell 40 to encompass the optical fibers 46 and to extrude through hole 70. Next, as shown in FIG. 5c, the protruding optical fibers and epoxy 60 is machined to form a smooth uniform surface. Preferably, the exiting optical fibers 46 faces perpendicular to the resulting ablation face 48.

The concepts of off-center guidewire lumen and a fiber bundle not pointed along the longitudinal axis as described above can be combined in other patterns. If one starts with a solid cylinder (approximating a catheter tip) and cuts out a section of the cylinder with one secant cut parallel to the longitudinal axis extending approximately the distance equal to the cylinder diameter, and one transverse cut perpendicular to the longitudinal axis, a flat secant surface remains at the end of the cylinder. Letting the cylinder represent the catheter tip, the guidewire lumen is placed off-center on the crescent surface at the tip as before. On the secant surface, opposite the guidewire lumen, the fiber bundle is disposed. This shape may be reminiscent of a lathe tool used to cut radial surfaces.

In a related embodiment, a fluted surface similar to the shape of cutting flutes on a ball-shaped end-mill is used. In such a surface on an ELCA catheter, the fibers are disposed on a curved surface, such that at the distal end, the fibers face forward along the longitudinal axis. At the proximal side of the fiber bundle, the fibers face to the side, pointed away from the longitudinal axis.

Preferably, the above described embodiments allow the catheter tip to ablate partially into the lesion tissue, and to ablate the tissue as the catheter is rotated while advancing through the lesion. The ablation front is not perpendicular to the longitudinal motion of the catheter, but rather is preferably perpendicular to the rotational motion of the catheter. This allows for more efficient ablation as the catheter is advanced and rotated, and reduces the amount of extraordinary precision required by the operator.

In another embodiment, untwisting the guidewire from the catheter is accomplished by stopping the circular motion periodically and reversing the motion. In another embodiment, a motorized handle is placed on the catheter shaft to rotate the shaft at a predetermined speed. The direction can be chosen so that it allows the tip to ablate while the laser is operating. The handle can also be synchronized to the rotational motion of the catheter with laser operation by providing a signal or switch closure to the laser system that causes the laser to operate in synchrony with the catheter motion.

In another aspect, various types of motors are employed in such a handle, such as a battery-powered electric motor, a pneumatically-powered air turbine, or a mechanically-powered mechanism such as an escapement or governor-controlled spinner. The motor can be designed to turn in one direction for a predetermined number of turns or amount of time, followed by an “unwind” motion in the opposite direction.

Avoiding the effect of wrapping the guidewire around the catheter can be accomplished by moving both the guidewire and the catheter simultaneously. If a round sleeve with two lumens is employed, one lumen disposed around the catheter shaft and the other disposed around the guidewire, then turning the sleeve would rotate both the guidewire and the catheter in synchrony. Both the catheter and the guidewire would turn inside the guide catheter and inside the artery being treated. This sleeve could be grasped by the motorized handle mentioned above, or by the physician's fingers. The sleeve might be fitted with slots that allow it to be quickly mounted onto the catheter shaft and removed. It could be clamshell design. Or it could be preloaded on the catheter shaft.

While the present invention has been particularly shown and described with reference to the foregoing preferred and alternative embodiments, it should be understood by those skilled in the art that various alternatives to the embodiments of the invention described herein may be employed in practicing the invention without departing from the spirit and scope of the invention as defined in the following claims. It is intended that the following claims define the scope of the invention and that the method and apparatus within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

What is claimed is:

1. A laser catheter tip comprising:
an outer shell having an axial direction and a radial direction;
an optical face; and
a light cable that transmits light from a source to the optical face;
wherein the optical face directs light from the light cable in at least a direction different from an axial direction of the outer shell.

2. The laser catheter tip according to claim 1, wherein the outer shell has a marker band disposed thereon for fluoroscopic visualation.

3. The laser catheter tip according to claim 1, further comprising a guidewire lumen attached to the outer shell.

4. The laser catheter tip according to claim 2, further comprising a guidewire lumen that is attached to the outer shell at a position off-axis from a central axis of the outer shell.

5. The laser catheter tip according to claim 4, wherein the catheter tip further comprises a distal face positioned at a axially deep location along the axial direction of the outer shell, wherein the distal face has a shallow position proximate the guidewire lumen and a deep position distally located from the guidewire lumen.

6. The laser catheter tip according to claim 1, wherein the optical face directs light perpendicular to the axial direction.

7. The laser catheter tip according to claim 1, wherein the optical face tapers from a radial outer position of the outer shell to a radial inner position of the outer shell, wherein the radial inner position of the outer shell is located at an axially deep location with respect to the axial direction of the outer shell to direct at least some illumination in the axial direction.

8. The laser catheter tip according to claim 1, wherein the optical face further comprises:
a first optical face portion;
a second optical face portion;
wherein the first optical face portion is located at an axially shallow position on the outer shell;
wherein the second optical face portion is located at an axially deep position on the outer shell;
wherein the first optical face portion has a taper that is shallower than the second optical face portion;
wherein the second optical face portion directs at least some illumination more toward an axially forward position of the outer shell than does the first optical face portion.

9. An optical catheter assembly comprising:
a bifurcating adaptor;
a light conveying cable connected to the bifurcating adaptor that transmits light;
a guidewire lumen entering the bifurcating adapter;
a fiber optic catheter assembly exiting the bifurcating adapter and containing the light cable and guidewire lumen;
a laser catheter tip connected to a distal portion of the fiber optic catheter assembly with respect to the bifurcating adapter, wherein the laser catheter tip comprises:
an outer shell having an axial direction and a radial direction;
an optical face; and
a light cable that transmits light from a source to the optical face;
wherein the optical face directs light from the light cable in at least a direction different from an axial direction of the outer shell.

10. The fiberoptic catheter assembly according to claim 9, wherein the outer shell has a marker band disposed thereon for fluoroscopic visualation.

11. The fiber-optic catheter assembly according to claim 9, further comprising a guidewire lumen attached to the outer shell.

12. The fiber-optic catheter assembly according to claim 19, further comprising a guidewire lumen attached to the outer shell at a position off-axis from a central axis of the outer shell.

13. The fiber-optic catheter assembly according to claim 9, wherein the outer shell further comprises the distal face positioned at a axially deep location along the axial direction of the outer shell, wherein the distal face has a shallow position proximate the guidewire lumen and a deep position distally located from the guidewire lumen.

14. The fiber-optic catheter assembly according to claim 10 wherein the optical face directs light perpendicular to the axial direction.

15. The fiber-optic catheter assembly according to claim 9, wherein the optical face tapers from a radial outer position of the outer shell to a radial inner position of the outer shell, wherein the radial inner position of the outer shell is located at an axially deep location with respect to the axial direction of the outer shell to direct at least some illumination in the axial direction.

16. A method for removing a blockage in a blood passage comprising the steps of:
providing a fiber optic catheter assembly having a laser catheter tip that directs light in a direction different than an axial direction of the laser catheter tip;
positioning the laser catheter tip approximate the blockage;
rotating the laser catheter tip to direct light against portions of the blockage.

17. The method according to claim 16, wherein the step of rotating the laser catheter tip comprises rotating a bifurcating adapter.

18. The method according to claim 16, wherein the step of rotating the laser catheter tip further comprises rotating a guidewire lumen attached to the laser catheter tip, wherein the guidewire lumen is attached to the laser catheter tip at a position off a central axis of the laser catheter tip.

19. The method according to claim 16, wherein the laser catheter tip has a marker band disposed thereon for fluoroscopic visualation.

20. The method according to claim 16, wherein the laser catheter tip comprises the laser catheter tip, wherein the outer shell has a marker band disposed thereon for fluoroscopic visualation.

21. A method for making a laser catheter tip, the method comprising the steps of:
positioning a plurality of optical fibers through an outer shell, wherein the outer shell has an axial direction and a radial direction;
directing the plurality of optical fibers in an off axis direction;
injecting an epoxy into the outer shell to affix the optical fibers in the outer shell; and
machining the plurality of fiber cables directed in the off axis direction to form an optical face that directs illumination at least in the off axis direction.
